(19) United States
(12) Patent Application Publication
(10) Pub. No.: US 2017/0218007 A1
(43) Pub. Date: Aug. 3, 2017

(54) A PROCESS FOR THE PREPARATION OF NUCLEOSIDE PHOSPHORAMIDATE

(71) Applicant: LUPIN LIMITED, Mumbai (IN)

(72) Inventors: Bhairab Nath ROY, Pune, Maharashtra (IN); Girij Pal SINGH, Pune, Maharashtra (IN); Dhananjai SHRIVASTAVA, Pune, Maharashtra (IN); Kishor Gulabrao MEHARE, Pune, Maharashtra (IN); Vineet MALIK, Pune, Maharashtra (IN); Sharad Chandrabhan DEOKAR, Pune, Maharashtra (IN); Abhijeet Avinash DANGE, Pune, Maharashtra (IN)

(21) Appl. No.: 15/500,620

(22) PCT Filed: Jul. 31, 2015
(86) PCT No.: PCT/IB2015/055828

§ 371 Date: Jan. 31, 2017

(30) Foreign Application Priority Data
Aug. 1, 2014 (IN) 2479/MUM/2014

(51) Int. Cl. C07H 19/10; C07F 9/58; C07F 9/653

(52) U.S. Cl. CPC C07H 19/10; C07F 9/653; C07F 9/585

(57) ABSTRACT

The present invention pertains to process for preparing nucleoside phosphoramidate and its intermediate. The present invention provides novel intermediate, its process for preparation and its use for the preparation of Sofosbuvir.

### FIELD OF THE INVENTION

The present invention pertains to process for preparing nucleoside phosphoramidates and their intermediates. Phosphoramidates are inhibitors of RNA-dependent RNA viral replication and are useful as inhibitors of HCV NS5B polymerase, as inhibitors of HCV replication and for treatment of hepatitis C infection in mammals. One of the recently approved phosphoramidate by USFDA is Sofosbuvir [1190307-88-0]. Sofosbuvir is a component of the first all-oral, interferon-free regimen approved for treating chronic hepatitis C. The present invention provides novel intermediate, its process for preparation and use for the preparation of Sofosbuvir. The present invention also gives one pot process for preparation of Sofosbuvir.

### BACKGROUND OF THE INVENTION

Hepatitis C virus (HCV) infection is a major health problem that leads to chronic liver disease, such as cirrhosis and hepatocellular carcinoma, in a substantial number of infected individuals. There are limited treatment options for individuals infected with hepatitis C virus. The current approved therapeutic option is the use of immunotherapy with recombinant interferon-[alpha] alone or in combination with the nucleoside analog ribavirin.

U.S. Pat. No. 7,964,580 (580) is directed towards novel nucleoside phosphoramidate prodrug for the treatment of hepatitis C virus infection.

US '580 patent claims Sofosbuvir and process for preparation of Sofosbuvir of Formula 1.

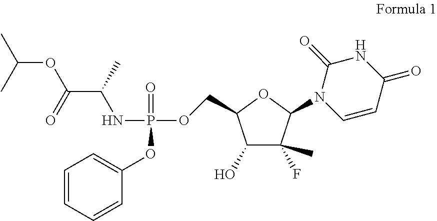 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

Process for preparation of Sofosbuvir as per US '580 patent involve reaction of compound of Formula 4″ with a nucleoside 5′

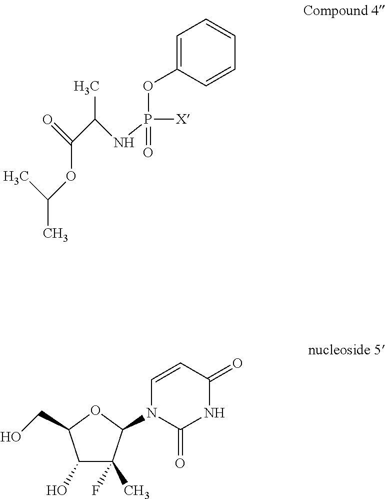

Wherein X′ is a leaving group, such as Cl, Br, I, tosylate, mesylate, trifluoroacetate, trifluroslfonate, pentafluorophenoxide, p-nitro-phenoxide.

### OBJECTS OF THE INVENTION

The object of the present invention is to provide a novel intermediate of Formula 2

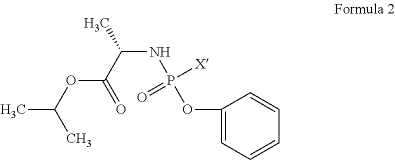

wherein X′ is a leaving group selected from 1-hydroxybenzotriazole, 5-(Difluoromethoxy)-1H-benzimidazole-2-thiol, 2-Mercapto-5-methoxybenzimidazole, cyanuric acid, 2-oxazolidinone, 2-Hydroxy Pyridine. The above leaving group can be optionally substituted with n-alkyl, branched alkyl, substituted alkyl; cycloalkyl; halogen; nitro; or aryl, which includes, but not limited to, phenyl or naphthyl, where phenyl or naphthyl are further optionally substituted with at least one of C₁-C₆ alkyl, C₂-C₆ alkenyl, C₂-C₆ alkynyl, C₁-C₆ alkoxy, F, Cl, Br, I, nitro, cyano, C₁-C₆ haloalkyl, C₁-C₆ acylamino, —NHSO₂C₁-C₆ alkyl, —SO₂N(R^{1′})₂, COR^{1″}, and —SO₂C₁-C₆ alkyl; (R^{1′} is independently hydrogen or alkyl, which includes, but is not limited to, C₁-C₂₀ alkyl, C₁-C₁₀ alkyl, or C₁-C₆ alkyl, R¹″ is —OR¹ or —N(R^{1′})₂).

Another object of the present invention is to provide a process to prepare the intermediate of Formula 2.

Another object of the present invention is use of the intermediate of Formula 2 in the preparation of Sofosbuvir of Formula 1.

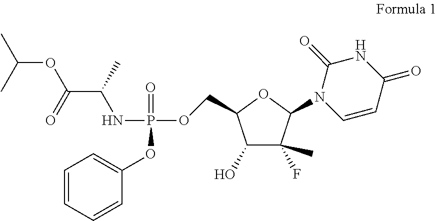 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

### SUMMARY OF INVENTION

One of the embodiments of the present invention is the novel intermediate of Formula 2.

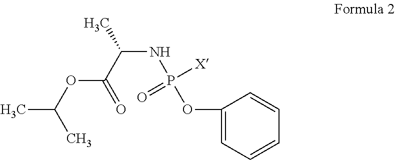

wherein X′ is a leaving group selected from 1-hydroxybenzotriazole, 5-(Difluoromethoxy)-1H-benzimidazole-2-thiol, 2-Mercapto-5-methoxybenzimidazole, cyanuric acid, 2-oxazolidinone, 2-Hydroxy Pyridine. The above leaving group can be optionally substituted with n-alkyl, branched alkyl, substituted alkyl; cycloalkyl; halogen; nitro; or aryl, which includes, but not limited to, phenyl or naphthyl, where phenyl or naphthyl are further optionally substituted with at least one of C₁-C₆ alkyl, C₂-C₆ alkenyl, C₂-C₆ alkynyl, C₁-C₆ alkoxy, F, Cl, Br, I, nitro, cyano, C₁-C₆ haloalkyl, —N(R^{1′})2, C₁-C₆ acylamino, —NHSO₂C₁-C₆ alkyl, —SO₂N(R^{1′})₂, COR^{1″}, and —SO₂C₁-C₆ alkyl; (R^{1′} is independently hydrogen or alkyl, which includes, but is not limited to, C₁-C₂₀ alkyl, C₁-C₁₀ alkyl, or C₁-C₆ alkyl, R¹″ is —OR¹ or —N(R^{1′})₂).

It is contemplated that compound of the Formula 2 is racemic because of the chirality at phosphorous. The phosphorous atom is chiral and that it has a corresponding Cahn-Ingold-Prelog designation of “R” or “S” which has their accepted plain meanings. It is understood that the chirality at phosphorous atom is present unless the substituents bound to the phosphorous exclude the possibility of chirality at phosphorous. Applicants contemplate use of the racemate and/or the resolved enantiomers.

Although the above structure of Formula 2 does not specifically depict chirality at phosphorus, the inventors recognize that stereochemical configurations are possible. Therefore, the compound of Formula 2 includes all possible stereochemical configurations possible for phosphorus.

In another embodiment of the invention Formula 2 can be prepared by reacting phosphorochloridate with S-oxazolidinone, R-oxazolidinone, benzimidazole, benzotriazole, triazine or 2-hydroxy pyridine or their derivatives.

In another embodiment of the present invention is disclosed a novel process for preparation of Sofosbuvir comprising reaction of novel intermediate of Formula 2 with 1-((2R,3R,4R,5R)-3-fluoro-4-hydroxy-5-(hydroxymethyl)-3-methyltetrahydro furan-2-yl) pyrimidine-2,4(1H,3H)-dione (Formula 3).

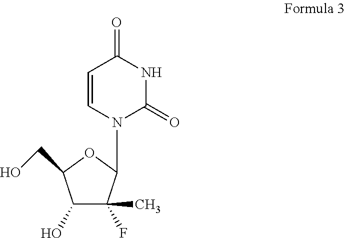 [SMILES: C[C@]1(F)C(n2ccc(=O)[nH]c2=O)O[C@H](CO)[C@H]1O]

### DETAIL DESCRIPTION OF THE INVENTION

According to the one embodiment of present invention, there is provided a novel intermediate of Formula 2

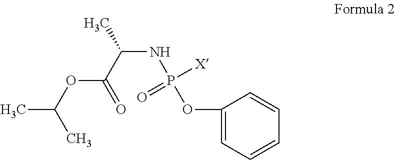

wherein X′ is a leaving group selected from 1-hydroxybenzotriazole, 5-(Difluoromethoxy)-1H-benzimidazole-2-thiol, 2-Mercapto-5-methoxybenzimidazole, cyanuric acid, 2-oxazolidinone, 2-Hydroxy Pyridine. The above leaving group can be optionally substituted with n-alkyl, branched alkyl, substituted alkyl; cycloalkyl; halogen; nitro; or aryl, which includes, but not limited to, phenyl or naphthyl, where phenyl or naphthyl are further optionally substituted with at least one of C₁-C₆ alkyl, C₂-C₆ alkenyl, C₂-C₆ alkynyl, C₁-C₆ alkoxy, F, Cl, Br, I, nitro, cyano, C₁-C₆ haloalkyl, —N(R^{1′})2, C₁-C₆ acylamino, —NHSO₂C₁-C₆ alkyl, —SO₂N(R^{1′})₂, COR^{1″}, and —SO₂C₁-C₆ alkyl; (R^{1′} is independently hydrogen or alkyl, which includes, but is not limited to, C₁-C₂₀ alkyl, C₁-C₁₀ alkyl, or C₁-C₆ alkyl, R¹″ is —OR¹ or —N(R^{1′})₂).

It is contemplated that compound of the Formula 2 is racemic because of the chirality at phosphorous. The phosphorous atom is chiral and that it has a corresponding Cahn-Ingold-Prelog designation of “R” or “S” which has their accepted plain meanings. It is understood that the chirality at phosphorous atom is present unless the substituents bound to the phosphorous exclude the possibility of chirality at phosphorous. Applicants contemplate use of the racemate and/or the resolved enantiomers.

Although the above structure of Formula 2 does not specifically depict chirality at phosphorus, the inventors recognize that stereochemical configurations are possible. Therefore, the compound of Formula 2 includes all possible stereochemical configurations possible for phosphorus.

According to yet another embodiment of present invention there is provided a process Formula 2 by reacting phosphorochloridate with S-oxazolidinone, R-oxazolidinone, benzimidazole, benzotriazole, triazine or 2-hydroxy pyridine or their derivatives such as 1-hydroxybenzotriazole, 5-(Difluoromethoxy)-1H-benzimidazole-2-thiol, 2-Mercapto-5-methoxybenzimidazole, cyanuric acid, 2-oxazolidinone, 2-hydroxy-3-nitro-5-(trifluoromethyl) pyridine, 2-Hydroxy-5-fluoropyridine 2-Hydroxy-5-nitropyridine 2-Hydroxy-3, 5-dinitropyridine.

According to yet another embodiment of present invention there is provided a process for preparation of Sofosbuvir comprising reaction of novel intermediate of Formula 2 with 1-((2R,3R,4R,5R)-3-fluoro-4-hydroxy-5-(hydroxymethyl)-3-methyltetrahydro furan-2-yl) pyrimidine-2,4(1H,3H)-dione (Formula 3).

Compound of Formula 3 can be prepared by methods known in the literature

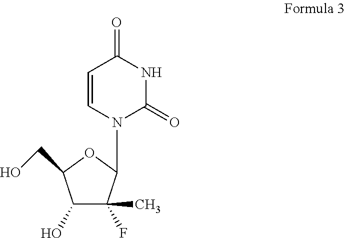 [SMILES: C[C@]1(F)C(n2ccc(=O)[nH]c2=O)O[C@H](CO)[C@H]1O]

### Example 1

Process for the Preparation of S-Oxazolidinone Derivative of Formula 2

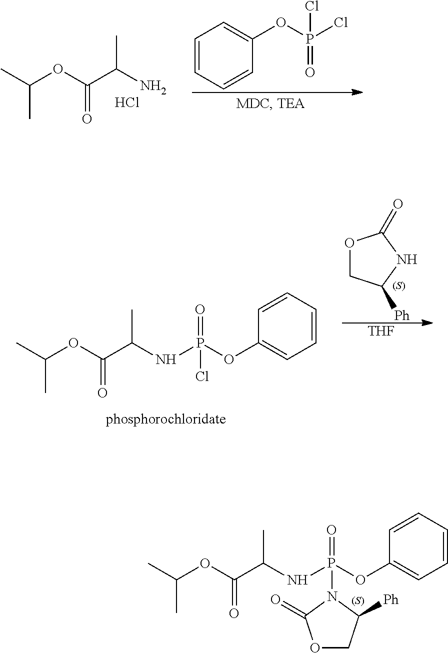

Step-1 Preparation of Phosphorochloridate Solution:

Dichloromethane (DCM 400 ml) was charged in round bottom flask flushed with nitrogen. Phenyl phosphodichloridate (18.30 ml) was added in one portion in the flask. The flask was cooled to −60°−70° C. with a dry ice-acetone bath. Solution of L-alanine isopropyl ester hydrochloride (20.6 gm)) in DCM (50 ml) was added to the reaction flask. To this was added a solution of triethylamine (11.20 ml) in MDC (100 ml) was added over a course of 60 minutes, while maintaining internal temperature below −70° C. throughout the addition. After completion of reaction, temperature of reaction mass was raised to room temperature.

100 ml THF was charged in another round bottom flask flushed with nitrogen followed by the addition of S-4-phenyloxazolidnone (10 gm). Triethyl-amine (11.2 ml) & LiCl (2.85 gm) were added to the above flask. The reaction mass was stirred for 15-30 min at room temperature and was cooled to 0-5° C. Phosphorochloridate solution from step-1 was added drop-wise to—the reaction flask in 15-45 min maintaining reaction temperature at 0-5° C. The reaction mass was stirred for 30-60 min at 0°−5° C. The reaction progress was monitored on thin layer chromatography. After completion of the reaction, the reaction temperature was raised to room temperature. Agitation was resumed for an additional 30 min. The reaction mass was filtered and concentrated under reduced pressure. To this was added diisopropyl ether (400 ml) and aqueous saturated ammonium chloride solution and reaction mass was stirred for 10-15 minutes. Organic layer was separated and was washed with water (100 ml) & dried over sodium sulfate and concentrated under vacuum. Cyclohexane (50 ml) was charged to the obtained oily mass and reaction mass was stirred till solid precipitated out. Solid was filtered and washed with cyclohexane and dried under vacuum (8.80 gm MP 56.5°-56.6° C.). The obtained product was characterized by mass, NMR & IR. ¹H NMR (DMSO-d₆) δ 1.142-1.18 (m, 9H), 3.85-3.92 (m, 1H), 4.72-4.89 (m, 2H), 5.31-5.32 (d, 1H), 6.25-6.3 (m, 1H), 6.95-7.31 (m, 10H); MS, m/e 433 (M+1)

### Example 2: Process for the Preparation of 2-Hydroxy Pyridine Derivatives of Formula 2

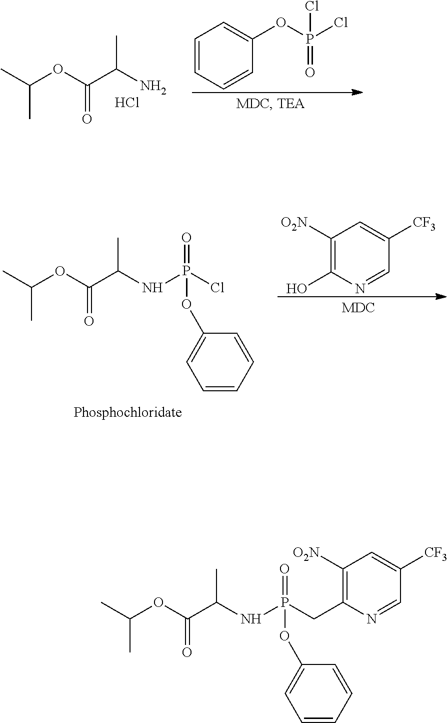

Anhydrous dichloromethane (DCM) 700 ml was charged in round bottom flask flushed with nitrogen. The flask was cooled to −60° to −70° C. in a dry ice acetone bath. Phenyl phosphodichloridate (76.04 gm) was added in one portion in the flask at −65° C. Solution of L-alanine isopropyl ester hydrochloride (60.56 gm) in DCM (50 ml) was added to the reaction mass. Solution of triethylamine (72.44 gm) in DCM (50 ml) was added to the reaction mass over a course of 60 minutes, while maintaining internal temperature below −70° C. throughout the addition. The resulting white slurry was agitated for additional 60 minutes. Then the temperature of reaction mass was raised to room temperature. Reaction mass was stirred for 60 min & TLC was checked. Reaction mass was filtered and rinsed with anhydrous dichloromethane (2×100 mL). The filtrate was concentrate under vacuum to 20 V and reaction mass was filtered, washed with DCM (15 ml). The filtrate was transferred to RBF. The reaction mass was cooled to 0°-10° C. A solution of 2-hydroxy-3-nitro-5-(trifluoromethyl) pyridine (15 gm) in DCM (100 ml) & triethyl amine (21.89 gm) was added to the reaction mass. Temperature of reaction mass was raised to 20-30° C. Reaction mass was stirred overnight. Reaction was monitored using TLC. After completion, the reaction mass was filtered and washed with DCM (30 ml). Filtrate was washed with water (150 ml×2). Organic layer was concentrated under vacuum and degased. Diisopropyl ether (200 ml) was charged to reaction mass and reaction mass was stirred for 15 minutes, filtered and washed with methyl ter-butyl ether (MTBE 30 ml). Filtrate was concentrated under vacuum and dried. (8.68 gm, MP−125.5°-131.5° C.). Obtained compound was characterized by Mass, NMR & IR. ¹H NMR (DMSO-d₆) δ 1.07-1.27 (m, 9H), 4.04-4.11 (m, 1H), 4.73-4.79 (m, 1H), 6.76-7.43 (m, 5H), 9.00-9.02 (d, 2H); MS, m/e 478 (M+1)⁺; FTIR, 1203, 1409, 1580, 1732, 3217.

Other 2-hydroxy pyridine derivatives of Formula 2 were prepared by following the process disclosed in example 2—

2-Hydroxy-5-fluoropyridine derivative of Formula 2; —¹H NMR (DMSO-d₆) δ 1.09-1.23 (m, 9H), 3.02-3.06 (m, 1H), 3.85-4.01 (m, 1H), 4.79-4.87 (m, 1H), 6.4-6.52 (m, 1H), 7.10-7.89 (m, 6H); MS, m/e 383 (M+1)⁺,

2-Hydroxy-5-nitropyridine derivative of Formula 2: —¹H NMR (DMSO-d₆) δ 1.06-1.22 (m, 9H), 4.0-4.02 (m, 1H), 4.7-4.8 (m, 1H), 6.5-6.6 (m, 1H), 7.12-7.42 (m, 6H), 8.66-8.68 (d, 1H), 9.07-9.13 (d, 1H); MS, m/e 410 (M+1)⁺

2-Hydroxy-3, 5-dinitropyridine derivative of Formula 2: —¹H NMR (DMSO-d₆) δ 1.11-1.24 (m, 9H), 3.04-3.09 (m, 1H), 4.8-4.86 (m, 1H), 7.09-7.39 (m, 5H), 8.97-9.06 (d, 2H)

### Example 3: Process for the Preparation of Sofosbuvir by Coupling of isopropyl(((3-nitro-5-(trifluromethyl)pyridin-2-yl)oxy)phenoxy)phosphoryl-L-alaninate with 1-((2R,3R,4R,5R)-3-fluoro-4-hydroxy-5-(hydroxymethyl)-3-methyltetrahydrofuran-2-yl)pyrimidine-2,4(1H,3H)-dione

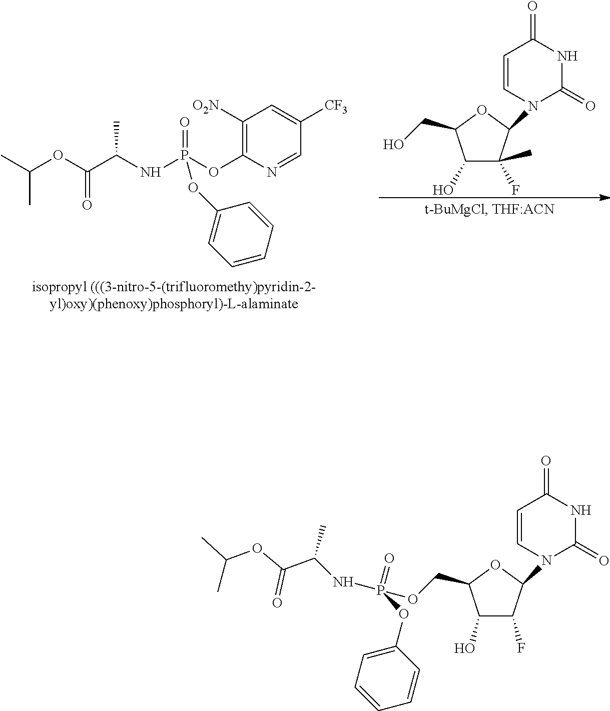 [SMILES: CC(C)OC(=O)[C@H](C)NP(=O)(Oc1ccccc1)Oc1ncc(C(F)(F)F)cc1[N+](=O)[O-] ; C[C@@]1(F)[C@H](O)[C@@H](CO)O[C@H]1n1ccc(=O)[nH]c1=O ; CC(C)OC(=O)[C@H](C)N[P@](=O)(OC[C@H]1O[C@@H](n2ccc(=O)[nH]c2=O)[C@H](F)[C@@H]1O)Oc1ccccc1]

To a solution of 1-((2R,3R,4R,5R)-3-fluoro-4-hydroxy-5-(hydroxymethyl)-3-methyltetrahydrofuran-2-yl)pyrimidine-2,4(1H,3H)-dione (0.2 gm) in THF (4 ml), tert-butylmagnesium chloride (0.80 ml, 1.7 M solution in THF) was added dropwise at room temperature and reaction mass was stirred for 30 minutes. A solution of pyridine derivative from example 2 (0.36 gm) in THF (4 ml) was added dropwise to the reaction mass at room temperature. Completion of reaction was monitored using TLC. After completion of reaction, reaction mass was quenched by using saturated ammonium chloride solution (10 ml). Reaction mass was extracted with ethyl acetate (50 ml). Organic layer was separated, dried over magnesium sulfate and concentrated under vacuum. The resulting residue was purified by column chromatography on silica gel & obtained solid product was characterized. MS, m/e 530.2 (M+1)⁺.

1. A compound of Formula 2 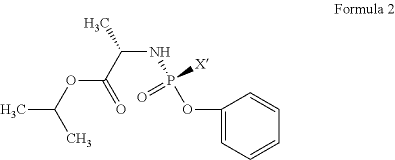
wherein X′ is selected from the group consisting of:
a) 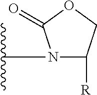
wherein R is substituted or un-substituted n-alkyl, branched alkyl, cycloalkyl; halogen; nitro; or aryl, wherein said aryl is optionally substituted with at least one of C₁-C₆ alkyl, C₂-C₆alkenyl, C₂-C₆alkynyl, C₁-C₆alkoxy, F, Cl, Br, I, nitro, cyano, C₁-C₆haloalkyl, —N(R^{1′})₂, C₁-C₆acylamino, —NHSO₂C₁-C₆ alkyl, —SO₂N(R^{1′})₂, COR^{1″}, and —SO₂C₁-C₆ alkyl; wherein R^{1′} is independently hydrogen or C₁-C₁₀alkyl; and R¹″ is —OR^{1′} or —N(R^{1′})₂;
b) 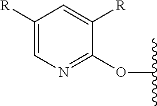
wherein R is substituted or un-substituted n-alkyl, branched alkyl, cycloalkyl; halogen; nitro or aryl, wherein said aryl is optionally substituted with at least one of C₁-C₆ alkyl, C₂-C₆alkenyl, C₂-C₆alkynyl, C₁-C₆alkoxy, F, Cl, Br, I, nitro, cyano, C₁-C₆haloalkyl, —N(R^{1′})₂, C₁-C₆acylamino, —NHSO₂C₁-C₆ alkyl, —SO₂N(R^{1′})₂, COR^{1″}, and —SO₂C₁-C₆ alkyl; wherein R^{1′} is independently hydrogen or C₁-C₂₀alkyl; and R¹″ is —OR^{1′} or —N(R^{1′})₂;
c) 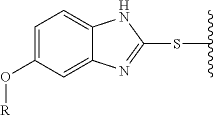
wherein R is H, NO₂, Cl, CHF2, or CF₃;
d) 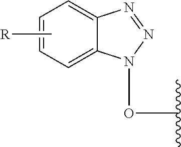
wherein R is H, NO₂, Cl, or CF₃;
and
e) 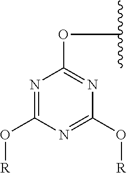
wherein R is hydrogen, substituted or un-substituted alkyl, or alkylaryl.

2. The compound according to claim 1, wherein said aryl is an optionally substituted phenyl or naphthyl.

3. A process for preparing the compound of Formula 2 as claimed in claim 1 comprising reacting a phosphorochloridate of following Formula 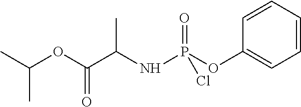 [SMILES: CC(C)OC(=O)C(C)NP(=O)(Cl)Oc1ccccc1]
with
a) 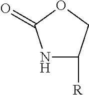
Wherein R is as defined in claim 1;
b) 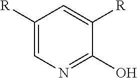
wherein R is as defined in claim 1;
c) 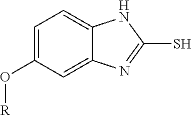
wherein R is as defined in claim 1;
d) 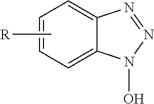
wherein R is as defined in claim 1; or
e) 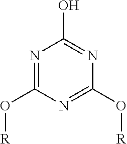
wherein R is as defined in claim 1.

4. The process of claim 3 wherein the phosphorochloridate of the Formula 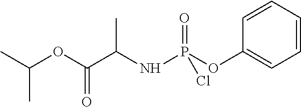 [SMILES: CC(C)OC(=O)C(C)NP(=O)(Cl)Oc1ccccc1]
is reacted with 1-hydroxybenzotriazole or 5-(Difluoromethoxy)-1H-benzimidazole-2-thiol, 2-Mercapto-5-methoxybenzimidazole; cyanuric acid; 2-oxazolidinone; 2-Hydroxy Pyridine; or a derivative thereof.

5. The process of claim 4 wherein the phosphorochloridate of the Formula 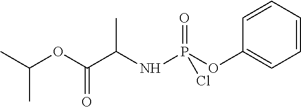 [SMILES: CC(C)OC(=O)C(C)NP(=O)(Cl)Oc1ccccc1]
is reacted with S-4-phenyloxazolidine; 2-hydroxy-3-nitro-5-(trifluoromethyl)pyridine; 2-Hydroxy-5-fluoropyridine; 2-Hydroxy-5-nitropyridine; or 2-Hydroxy-3,5-dinitropyridine.

6. A process for the preparation of Sofosbuvir comprising reacting a compound of Formula 2 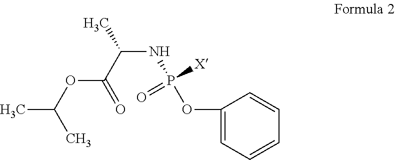
with a compound of Formula 3

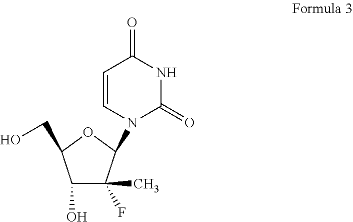 [SMILES: C[C@@]1(F)[C@H](O)[C@@H](CO)O[C@H]1n1ccc(=O)[nH]c1=O]

7. Use of compound of Formula 2 for the preparation of Sofosbuvir.
